(19) United States
(12) Patent Application Publication
(10) Pub. No.: US 2010/0316677 A1
(43) Pub. Date: Dec. 16, 2010

(54) USE OF BAMBOO LEAF TOTAL FLAVONES IN THE PREPARATION OF MEDICINE AND HEALTH FOOD FOR PREVENTION AND TREATMENT OF PROSTATIC DISEASES

(75) Inventors: Ying Zhang, Zhejiang Province (CN); Xiaoqin Wu, Zhejiang Province (CN); Yonghua Zhang, Zhejiang Province (CN); Huafang Cai, Zhejiang Province (CN); Boyi Lu, Zhejiang Province (CN)

Correspondence Address: HAMRE, SCHUMANN, MUELLER & LARSON, P.C., P.O. BOX 2902, MINNEAPOLIS, MN, 55402-0902, US

(73) Assignee: ZHEJIANG UNIVERSITY (HANGZHOU) LEAF BIO-TECHNOLOGY CO., LTD., Hangzhou (CN)

(21) Appl. No.: 10/558,130

(22) PCT Filed: May 24, 2004
(86) PCT No.: PCT/CN2004/000531

§ 371 Date: May 27, 2009

(30) Foreign Application Priority Data
May 25, 2003 (CN) 03128889.8

(51) Int. Cl. A61K 9/00; A61K 36/899

(52) U.S. Cl. 424/400; 424/750

(57) ABSTRACT

This invention discloses the new uses of Lophatherum Total Flavones in medical, pharmaceutical, and healthcare fields. Lophatherum Total Flavones have effects of anti-bacteria, inhibiting bacteria, anti-inflammation, anti-prostatic hyperplasia, anti-platelet aggregation, anti-tumor, and promotion of immunity, etc., with safety and without toxicity, suitable for chronic oral administration, and especially suitable for prevention and treatment of senile chronic degenerative diseases; Lophatherum Total Flavones may be used in fields of pharmaceutical and foods, as a natural drug for prevention and treatment of prostatitis, prostatic hyperplasia, and prostate cancer, or as a dietary supplement.

### TECHNICAL FIELD

This invention is directed to the field of medical technology. It relates to the Use of Bamboo Leaf Total (lophatherum) Flavones in the preparation of medicine and health food for Prevention and Treatment of Prostatic Diseases. The lophatherum total flavones have effects of anti-bacteria, inhibiting bacteria, anti-inflammation, anti-prostatic hyperplasia, anti-platelet aggregation, anti-tumor, and enhancing immunity, etc., and may be used as natural drugs for prevention and treatment of prostate diseases, or as dietary supplements of health foods.

### BACKGROUND ART

While the world turns into an aging society in the 21st century, significant changes occur in disease spectrum and medical pattern; good opportunities have been brought for development of natural drugs due to risks caused by synthetic drugs, and due to obvious benefits achieved by using traditional Chinese drugs all over the world; and now, traditional Chinese medicine has been recognized again by the world, and natural drugs are highly expected in “keeping healthy” or in “prevention and treatment”. Natural drugs and their preparations, by virtue of their safety, no side effects, mild actions, and adapting multiplicity, will be ideal medicines for senile patients with chronic diseases, especially diseases of multiple organs. In this point, the 21^{st} century is an era of natural drugs.

In recent 20 years, prostatic diseases are increasing and in younger people, and have been a common disease threatening male health. Prostatic diseases mainly consist of prostatitis and prostatic hyperplasia, and of gradually increasing prostate cancer.

The prostatitis syndrome (PS) is a well-recognized stubborn medical disease, known as an incurable disease other than the cancer. PS is a common, frequently occurring disease of male urinary system, accounting for 25% of urinary surgery clinic. The incidence of this disease is 24.3% in our country, and reported in literatures as 35-98% abroad. The two ages with peak incidence are of 30-39 and 60-69. Bennett et al. (1990) reported that the incidence was 73% in American, approximately 2 millions of patients each year, mainly with mixed acute and chronic inflammation of prostate; however, mainly with multi-focal chronic inflammation of prostate in our country. PS may affect 50% males in some periods of the whole life (Collins M M et al.) How common is prostatitis? A national survey of physician visits. J Urol 1998; 159:1224-1228; Roberts R O et al., A review of clinical and pathological prostatitis syndromes. Urology 1997; 49:809-821)).

Clinically, PS falls into four categories: acute bacterial prostatitis (ABP), chronic bacterial prostates (CBP), chronic non-bacterial prostatitis (NCP), and prostatic pain (PD). Among them, ABP and CBP amount for approximately 5%, NCP 64%, and PD 31% [Gerald J D et al. Prostatitis. Clin Microbiol Rev 1998; 11(4):604-613]. It is obvious that the chronic non-bacterial prostatitis (NCP) amounts for the majority of PS patients. PS is of complex etiology and pathology; current studies have suggested that its occurrence and development are closely related with multiple factors including microbial infection, intra-prostate urine return, and systemic and local immune function, etc.; however, its actual pathogenesis is still not known.

Presently, no anti-bacterial drug may clinically achieve good therapeutic effects for PS. The reason lies in the following aspects. First, the prostate has an particular anatomical site, in favor of access of bacteria in urethra to it, but not of its drainage, so that it is difficult to eliminate inflammatory secretion; secondly, the prostate has a specific structure with a fat layer in its prostatic epithelium, so that the anti-bacterial drug is difficult to disperse from blood plasma to its prostatic acinus, and moreover, affected by pH in prostate fluid, to obtain effective concentration for killing and inhibiting bacteria in the gland, the drug must be lipophilic, with high dissociation constant, low bonding rate to plasma proteins, low toxicity, and can be administered chronically, however, no anti-bacterial drugs have all these features in present clinical application. The pathological changes, i.e. prone to be fibrous around the focus, in the prostate itself, may often affect the dispersion of the drug to the lesion. And now, the miracle drug is in dire need in the market.

In treatment of PS, commonly used are antibiotics, including 5-fluoro quinolones, sulfanilamides, tetracyclines, or erythromycins, etc., up to 8-10 weeks of administration; chronic PS are predisposed to recur after withdrawal of antibiotics. Non-bacterial PS generally needs no antibiotics. Presently, there are many therapies of antibiotics in combination with local treatments, including intra-spermaductus injection, intra-prostatic local injection, injection around the prostate, and blockage around the prostate, which may achieve some effects, but cannot cure chronic PS. While many patients are complicated with many infections (meanwhile by chlamydia, mycoplasma, mildew and trichomoniasis, staphylococci, and resistant gonococci, etc.), it is difficult for many of the present antibiotics to fight with those pathogens at the same time. High doses of concurrent drugs have great damage to internal organs, and oral administration of high doses of antibiotics may easily result in poisoning hepatitis, poisoning nephritis, and impairment of gastrointestinal function. The selected drugs for intra-gland injection cannot yet kill several pathogens, and meanwhile, the sclerosing conglutination of mechanical injury caused by multiple injections, may result in permanent injury to patients, great difficulty to final treatment, and incurable sequela.

α-receptor blockers including tamsulosin hydrochloride and doxazosin may decrease urinary pressure, mitigate TPUR, relieve symptoms of patients with chronic PS, and may have better effects in concurrent administration with antibiotics. Doxazosin may be used in first-line therapy for NCP, and meanwhile, in auxiliary treatment of CBP. Non-steroidal anti-inflammatory agents such as ibuprofen may temporarily relieve painful symptoms and inflammation. Allopurinol may decrease the concentration of uric acid of whole body and of the prostate, eliminate active oxygen-free radicals, relieve inflammation, and mitigate pain. Moreover, oral administration of anticholinergics such as oxybutinin, of striated-muscular relaxants such as diazepam, of pollen extracts such as cenilton, and of zinc oral preparations, may be effective to some patients with PS.

The benign prostatic hyperplasia is one of common prostatic diseases in senile males. The current statistic results have demonstrated that this disease initially occurs in the age of 40 years, and highly in the age of 50-70 years. The result in a necropsy in foreign countries demonstrated that, more than half of 50-year-old males have BPH, and up to 75% of 70-year-old males have it. In the past, the disease occurred 10 years later in the ages of males in our country less than in the ages of those in occident countries, but gradually in younger ages of males in our country in recent years. In addition to extended average life, it is also associated with many factors from improvement of dietary level, and from stimulation by smoking and alcohol, or Yang-strengthening materials.

Prostate cancer (PC) is a common malignant tumor of males in occident countries. Its incident is the highest in American, with more than 35,000 yearly deaths, just secondary to lung cancer [Parker S L, Tong T, Bolden S, et al. Cancer Statistics. CA Cancer J Clin, 1997; 47:5-27]. The incidence of PC is much lower in our country than in Western countries, but with increase of the economic level, with change of life style, and with extended average life, the incidence of PC has gradually increased, and PC has occurred gradually in younger ages and been a significant disease threatening lives of senile males in our country. According to statistic data, the incidence of PC has increased from 2,000/100,000 people in 1950s to 12,000-34,000/100,000 people in 1990s, and has been presently the third in urinary tumors.

The difference between the incidences of PC in Eastern and Western countries, has drawn great attention of medical and dietetic professionals, and extensive studies have been conducted in succession; however, its actual pathogenesis is not clearly known up to now, and still in stage of investigation. PC is of complex pathogeny, affected by ages, hereditary factors, and environmental factors. Epidemiological data have demonstrated, dietary and environmental factors may affect more significantly the occurrence of PC than hereditary factors. Large amount of fruits and vegetables are contained in diets of Eastern people with low incidence of PC. However, those miraculous ingredients of food have not been determined up to now. Presently there are many therapies involving surgical operation, hormones, and chemical drugs, etc., but no one may significantly increase the survival of patients with PC, and moreover, many patients have lost the optimal opportunities for surgery when confirmed in diagnosis. Therefore, natural chemical drugs (especially phytochemical drugs) for prevention are of great significance in dietary interference to reduce or slow the development of PC.

Epidemiological data and clinical studies have found that, the consumption of tomato products may significantly decrease the risk of PC. A follow-up study involving 14,000 religionary males for 6 years, found that, lycopene taken not less than 5 times may significantly decrease the risk suffering from PC [Mills P K, Beeson W L, Phillips R L, et al. Cohort study of diet, life style and prostate cancer in Adventist men. Cancer, 1989; 64: 598-604]. A follow-up studies involving 48,000 healthcare professionals in America in 1986-1992, conducted by the research room of nutriology in Harvard Medical School [Giovannucci E L, Ascherio A, Rimm E B, et al. Intake of carotenoids and retinol in relationship to risk of prostate cancer. J Natl Cancer Inst, 1995; 87:1767-1776], also demonstrated that, there is a negative correlation between the ingestion of large amount of lycopene-rich products and the risk of PC.

In traditional Chinese medicine, prostatic diseases fall into categories of “dysuria”, “whitish discharge from the urethra”, “stranguria due to overstrain”, and “retention of urine”, etc. Analysis of conditions and treatment of diseases according to their categories in traditional Chinese medicine: patients with damp-heat accumulated for a long time, suitable for clearing away heat-evil, expelling superficial evils, relieving dysuria, and treating stranguria; patients with Yin deficiency accompanied with flaming-up of evil fire, suitable for nourishing Yin, clearing away heat-evil, relieving dysuria, and treating stranguria; patients with renal-Qi deficiency, suitable for nourishing kidney and arresting seminal discharge, assisted by relieving dysuria and treating stranguria; and patients with Qi stagnation and blood stasis, suitable for activating the blood circulation, eliminating blood stasis, and promoting the flow of Qi and relieving its stagnation. Many physicians of traditional Chinese medicine have different analysis and categories for prostatic diseases, thus resulting in different uses of drugs; most of present studies lack random controls, strict clinical observation and therapeutic evaluation, and thus limit the application of traditional Chinese drugs in treatment of prostatic diseases.

Due to the particular anatomical structure of the prostate, with long and narrow duct, pathogenic microorganism is difficult to be eliminated and cleared away once it enters the prostate. Due to barrier between prostate and blood, drugs are difficult to disperse to the prostate through the fat membrane of prostate epithelium, and to reach effective concentration. Delay in treatment and resistance to antibiotics may result in chronic inflammation that may result in narrowing of duct lumen and hyperplasia of fibrous tissues, so that microorganism are left in prostatic tissue, and thus chronic focus is produced, leading to low recovery and high recurrence. In the other hand, the pathogeny and pathogenesis of PS are not clearly known, its clinical signs are readily overlapping with those of prostatic hyperplasia and prostate cancer, leading to difficulty and uncertainty of clinical diagnosis; up to now, there are no rational and effective therapy and drugs for chronic PS. In October 2000, the NIH-IPCN conference suggested that, in treatment of chronic PS, antibiotics, α-receptor blockers, and anti-inflammatory drugs are used as first-line therapy, physical therapy (including microwave), phytochemical preparations as second-line therapy, and finasteride and pentose polysulfate as third-line therapy.

In the Clinical Guideline of New Traditional Chinese Drugs, constituted by the Ministry of Public Health, chronic PS falls into four primary categories, i.e. morbid leukorrhea due to damp-heat, stagnation of Qi and blood stasis, deficiency of Yin of the liver and the kidney, and deficiency of Yang of the kidney. Chinese patent medicines including Qiliexie Pills, Huixiang Juhe Pills, Liuwei Dihuang Pills, Shengqi Pills, and Yejuhua Suppositories, etc., may mitigate symptoms of PS to different extents. Modern studies have demonstrated that, medicines for activating blood circulation and removing blood stasis, including Radix Salviae Miltiorrhizae, Radix Achyranthis Bidentatae, and Semen Vaccariae, may relieve edema at the site of the focus, eliminate inflammatory obstruction, dredge prostatic duct, soften fibrous tissue, and increase local blood flow, so that the drug is ready to reach the focus, and increase drug concentration; medicines for relieving dysuria and removing toxicity, including herba patriniae and crude semen coicis, have effects of inhibiting bacteria, removing inflammatory focus, promoting elimination of inflammatory secretion, increasing immunity, and enhancing repair of tissues. The concurrent administration of traditional Chinese drugs and Western drugs may get twice the effects with half the effort.

Based on the ages of more than 50 years when the disease occur, and on the particular anatomical sites of the prostate, it is considered that, in traditional Chinese medicine, the occurrence of PC is mainly due to deficiency of vital Qi and interference by damp-heat and evil toxicity, leading to maladjustment of Yin and Yang, disorders of internal organs, and disorders of Qi and blood circulation, resulting in mutual combination of blood stasis, phlegm, and evil toxicity, and thus forming tumor. According to the change of pathogeny of PC and the change of falsehood and reality of symptoms, the early stage with accumulation of evil toxicity is suitable for relieving heat and removing toxicity; the moderate stage with mutual combination of phlegm and stasis is suitable for reducing phlegm, softening core, and reducing masses and nodulations; and the later stage with deficiency of vital Qi, and of both Yin and Yang, is suitable for nourishing Qi and blood, tonifying Yin, and reconciliating Yang. According to clinical practice, common internal therapies include: clearing away heat, removing dysuria, activating blood, eliminating stasis, nourishing Yin and Yang, removing toxic substances, regulating Qi, removing retained fluid and phlegm, and arresting discharges. Bamboo leaf (herba lophatheri) is a medicine used in common formulations, for removing heat, eliminate toxicity, and inducing diuresis, for example, Xiaoji drink in “Jisheng Recipe”. Based on records in Big Dictionary of Traditional Chinese Herbs, action and indications of herba lophatheri include: removing heat, easing the mind, promoting the production of body fluid, and causing diuresis; indicated in fidgetness and thirst in febrile diseases, infantile convulsion, cough and hematemesis, red complexion, scanty dark urine, aphthous stomatitis, and boil of the tongue, etc. In 1998, herba lophatheri is enrolled in the list of natural material for uses as both a drug and food. In recent years, studies have demonstrated that, the bamboo leaves of Phyllostachys Sieb. et Zucc, represented by Phyllostachys nigra var. hnonis (Bean) Stepf ex Rendle, contain large amount of flavones, lactones, hydroxybenzene acids, anthraquinone, polysaccarides, specific amino acids, aromatic ingredients, and trace elements such as manganese, zinc, selenium, etc., and have multiple physiological and pharmacological effects.

In the view of lacking effective therapeutic drugs for prostatic diseases, the development of effective drugs from natural plants is in urgent need for treatment of prostatic disease.

### SUMMARY OF THE INVENTION

The object of this invention is to provide Lophatherum Total Flavones used as a natural drug and/or a supplement of food, for the prevention and treatment of prostatic diseases.

In the first aspect of this invention, it provides a use of Lophatherum Total Flavones in medicine and health food as a factor for prevention and treatment of prostatic diseases, wherein the use is in the manufacture of medicine and health food for prevention and treatment of prostatic diseases.

In one preferred embodiment, the prostatic diseases comprise prostatitis, prostatic hyperplasia, and prostate cancer.

In one preferred embodiment, the medicine and health food comprise Lophatherum Total Flavones and a substance for prevention and treatment of prostatic diseases which is selected from the group consisting of: antibiotics, α-receptor blockers, anti-inflammatory drugs, and plant extracts. More preferably, the plant extracts are selected from the following group: vegetable polysaccharides, vegetable flavones, vegetable sterols, pollen extracts, and lycopene.

In one preferred embodiment, the forms of medicine and health food product are tablets, capsules, granules, pills, droplets, pre-emulsion, micro-emulsion, suspension, syrup, various enteric-coated forms, injections, sprays, ointments, or preparations for intrarectal administration such as suppositories, etc.

In one preferred embodiment, the dose of Lophatherum Total Flavones (content of total flavones glycoside, calculated on rutin) is 10-1000 mg/day for adults, once or twice per day. More preferably, the preferred dose of Lophatherum Total Flavones (content of total flavones glycoside, calculated on rutin) is 50-600 mg/day for adults, once or twice per day.

In one preferred embodiment, the plant extracts are selected from the group consisting of vegetable polysaccharides, vegetable flavones, vegetable sterols, pollen extracts, and lycopene.

In one preferred embodiment, the medicine and health food comprise 0.01-99 wt % of Lophatherum Total Flavones.

In one preferred embodiment, the medicine and health food comprise 0.1-90 wt % of Lophatherum Total Flavones.

### DESCRIPTIONS OF FIGURES

FIG. 1 showed the HPLC chromatogram of Lophatherum Total Flavones (EOB-f01) (of 4 main C-glycoside flavones).

FIG. 2 is the infrared chromatogram of Lophatherum Total Flavones (EOB-f01) (tabletted with potassium bromide).

FIG. 3 is the ultraviolet chromatogram of Lophatherum Total Flavones (EOB-f01) (dissolved in spectroscopically pure methanol).

FIG. 4 showed the representation of pathological slices of prostatic tissue from normal rats.

FIG. 5 is the representation of pathological slices of prostatitis tissue from rats by the dose of EOB-f01 400 mg/kg.

FIG. 6 is the representation of pathological slices of prostatitis tissue from rats by the dose of EOB-f01 200 mg/kg.

FIG. 7 showed the representation of pathological slices of prostatitis tissue from rats in the model group (by carrageenan).

### MODE FOR CARRYING OUT THE INVENTION

China is always known as the “Kingdom of Bamboo”, provided with extremely rich bamboo resources and with bamboo culture of long standing. In the domain of our country, there are more than 40 families of bamboo with more than 400 species of bamboo, approximately 4 million ha of bamboo forest. According to incomplete statistic data, there are more than 100 million people in our country who afford their life by bamboo forest or by processing of bamboo products. Bamboo is a significant part of forest resources, not only with high economic values but also with extensive ecological and social benefits. Bamboo, with its particular biological and ecological features, and for multiple uses, is gradually paid with attention by people, and plays a more and more important role in the sustainable development stratagem of China.

The investigation and development in active ingredients of bamboo in our country are at the leading level in the world. The bamboo leaf extract is a preparation of vegetable flavones developed in 1990s by Zhang Ying et al, and their patents of invention, i.e. “A Health Beer Supplemented with Lophatherum Flavones (ZL 98 1 04563.4)” and “the Method for Producing Extract or Powder of Flavonoids from Bamboo Leaves”, are authorized by the Patent Bureau of China in 2000 and 2001, respectively. Lots of studies have demonstrated that, Lophatherum Flavones have excellent biological effects of anti-free radicals, anti-oxidation, anti-aging, anti-bacteria, and anti-virus, of protecting cardiovascular and cerebrovascular systems, and of prevention and treatment of senile degenerative diseases, etc. Recently, it has cut a figure in the field of functional food and medical health food, with its rich resources, specific functional factors, believing safety, highly effective and stable preparation quality, and pure, fresh, sweet, and fragrant bamboo taste (Zhang Ying, A Natural and Functional Supplement—Lophatherum Extract, Fine and Special Chemical Substances, 2002, 10(7):20-22).

The functional factors in Lophatherum Total Flavones mainly include C-glycoside flavone, and the four Lophatherum C-glycoside flavones include orientin, homo-orientin, vitexin, and isovitexin. C-glycoside flavones compared with glycoside, have the following outstanding advantages: (1) stable structure, difficult to be degraded; (2) deeply dispersed into the site of focus, and directly exerting therapeutic effects; (3) increased hydrophilicity, in favor of development of drugs, foods, and cosmetics. C-glycoside flavones have been paid attention by international academic circle, and this field is the newest edge of study.

Flavonoids are extensive distributed in melons and fruits, vegetables, teas, soybeans and products thereof, and also the active ingredients of many Chinese herbal medicines. Bioflavonoids of natural source have small molecular weight, readily absorbed by human body, and permeated through blood-brain barrier. Now, the studies on their physiological effects are focused on: effects of anti-free radicals and anti-oxidation, correlation with cancer and internal secretion, anti-bacterial and anti-viral effects, and similar effects of estrogens, etc. However so far, public reports are rarely seen, of flavonoids directly associated with pathology of the prostate, and especially, there are no reports of effects of Lophatherum flavonoids associated with physiology and pharmacology of the prostate.

Based on the previous studies, Zhang Ying and her colleagues have conducted systemic studies on the relevant functions of Lophatherum Total Flavones (EOB-f), associated with pharmacology of the prostate, and found that Lophatherum Total Flavones may be very effectively used in treatment of prostatic diseases. Now it is concluded as follows:

1. Inhibition of Bacteria

1.1 Source of Strains

E. faecalis, S. pyogenes, S. epidermidis, P. vulgaris, and K. pheumoniae, are obtained among specimens of clinical urogenital system from Zhejiang People's Hospital, and separated 3 months prior this trial. The standard strains, i.e S. aureus (ATCC25923) and E. coli (ATCC25922) are provided by Zhejiang Center of Clinical Inspection.

1.2 Samples of Lophatherum Total Flavones

Product code: EOB-f01; content of standard total flavones glycoside ≧50%, experimental values 56.7%; lyophilized brown crystalline powder, purchased from Hangzhou Lifu Bio-science and Technology Co., Ltd., of Zhejiang University. Preserved in a desiccator; reconstituted with distilled water prior to use, assisted in dissolution by ultrasonic wave.

1.3 Experimental Method

It was carried out in accordance with the relevant contents in Operating Procedures of National Clinical Inspection by the Medical Department of the Ministry of Public Health, of People's Republic of China, edition 2.

1.4 Results

Infecting factors are generally considered as the predominant role in occurrence of acute and chronic bacterial prostatitis; however, with deeper investigation on the pathogeny, it has demonstrated that chronic non-bacterial prostatitis is also associated with bacterial infection. Presently, pathogenic gram-negative bacteria in bacterial prostatitis mainly include E. coli, as well as P. vulgaris, B. blue-pus, and K. pheumoniae, etc.; and pathogenic gram-positive bacteria mainly include S. aureus, as well as S. epidermidis, S. pyogenes, and gonococci, etc. The applicant selected 7 common species of pathogenic bacteria to urinary system, and conducted in-vitro trial for inhibition of bacteria, and the results demonstrated that EOB-f01 inhibited all the above-mentioned bacteria, although to a different extent; see Table 1.

TABLE 1
Inhibition of EOB-f01 against Common Pathogenic Bacteria
to Urinary System

| | Minimum inhibiting |
|---|---|
| Tested strains | concentration (MIC) |
| E. faecalis | 3.125 mg/ml |
| S. pyogenes | 3.125 mg/ml |
| S. epidermidis | 0.78 mg/ml |
| P. vulgaris | 1.56 mg/ml |
| K. pneumoniae | 25.0 mg/ml |
| S. aureus | 1.56 mg/ml |
| E. coli | 25.0 mg/ml |

2. Anti-Inflammation

2.1 Effects on Swelling in Ear Auricle of Mice, Caused by Croton Oil

Randomly assign mice into 5 groups, 12 mice in each group, as follows: \textcircled{1} Blank-controlled group: saline of equal volume, 0.8 mL/20 g; \textcircled{2} Positive-controlled group: indomethacin 10 mg/kg; \textcircled{3} High-dose group: 10 mg/mL EOB-f01, 400 mg/kg; \textcircled{4} moderate-dose group: 5 mg/mL EOB-f01, 200 mg/kg; \textcircled{5} Low-dose group: 2.5 mg/mL EOB-f01, 100 mg/kg. The animals received the administration through gastric irrigation (ig). once per day for 7 continuous days (for indomethacin, only once); in 1 h after the final dose, smear the right ear of the mice with 2% croton oil, 0.02 ml/mouse, cause inflammation, sacrifice them after 4 h, shear away their left and right ears, punch down their left and right ear slice with 9 mm trephine, and weigh them, taking the difference of weights between left and right ear slices as the swelling rate.

The experimental results demonstrated that, after gastric irrigation for 7 days, EOB-f01 200 mg/kg and 400 mg/kg have significant (p<0.05) and extremely significant (p<0.01) inhibition against ear swelling caused by croton oil in an obvious dose-dependent manner (Table 2).

TABLE 2
Effects of EOB-f01 on swelling of ear auricle of mice caused
by croton oil ( X̅ ± SD)

| | Dose | N | Swelling rate of ear |
|---|---|---|---|
| Groups | (mg/kg body weight) | (Count) | auricle(mg) |
| Blank-controlled | — | 13 | 19.8 ± 2.9 |
| group | | | |
| Indomethacin | 10 | 12 | 14.8 ± 5.1** |
| group | | | |
| High-dose group | 400 | 12 | 15.8 ± 2.7** |
| Moderate-dose | 200 | 12 | 16.9 ± 2.8* |
| group | | | |
| Low-dose group | 100 | 12 | 18.2 ± 3.9 |

*p < 0.05,
**p < 0.01, compared with the controlled groups.

2.2 Effects on Swelling in Ear Auricle of Mice, Caused by Xylene

Assign the mice into 5 groups according to their body weight, 10 mice in each group, as 2.1 section. Give the mice once per day through gastric irrigation (ig), for 7 days (indomethacin, given only once); in 30 min after the final dose, smear the right ear of the mice with xylene, 0.05 ml/mouse, cause inflammation, sacrifice them in 15 min, shear away their left and right ears, punch down their left and right ear slice with 9 mm trephine, and weigh them, taking the difference of weights between left and right ear slices as the swelling rate. Judge the effects compared with those of the controlled groups.

TABLE 3
Effects of EOB-f01 on swelling of ear auricle of mice
caused by xylene ( X̅ ± SD)

| | Dose | N | Swelling rate of ear |
|---|---|---|---|
| Groups | (mg/kg body weight) | (Count) | auricle (mg) |
| Blank-controlled | — | 10 | 11.4 ± 4.9 |
| group | | | |
| Indomethacin | 10 | 10 | 6.4 ± 2.3** |
| group | | | |
| High-dose group | 400 | 10 | 7.8 ± 2.2* |
| Moderate-dose | 200 | 10 | 11.1 ± 3.4 |
| group | | | |
| Low-dose group | 100 | 10 | 11.1 ± 3.0 |

*p < 0.05,
**p < 0.01, compared with the controlled groups.

The results in Table 3 showed that, after gastric irrigation of mice for 7 days, EOB-f01 400 mg/kg has significant (p<0.05) inhibition against the swelling of ear auricle caused by xylene, but has no obvious effects in moderate- and low-dose groups.

2.3 Effects on Prostatitis of Mice Caused by Carrageenan

Assign SD male rats with body weight 204-252 g, into 7 groups according to their body weight, 10 rats in each group: (1) controlled group: 1 mL/100 g saline of equal volume; (2) model group: 1 mL/100g of equal volume; (3) indomethacin group: 12 mg/kg (1.2 mg/mL); (4) high-dose group: EOB-f01 400 mg/kg (40 mg/mL); (5) moderate-dose group: EOB-f01 200 mg/kg (20 mg/mL); (6) low-dose group: EOB-f01 100 mg/kg (10 mg/mL). Give the rats through gastric irrigation once per day, for 7 days (indomethacin, only once prior to operation), and begin the operation in 0.5 h after the final dose.

Conduct anesthesia for the rats, then fix them in supine position, shear off hair at their abdomen, and sterilized by iodine tinture; under sterilized condition, cut 1.5 cm at the center of their abdomen, expose the abdominal part of prostate, inject rats in (2)-(6) groups with 1% carrageenan saline, 0.02 mL/rat, with No. 4.5 syringe, immediately return the prostate to the abdominal cavity, add 1 drop of penicillin in the abdominal cavity, and immediately suture the abdominal cavity by two-layer ways. Replace carrageenan with saline 0.02 mL/rat, for rat in the controlled group (1), and conduct pseudo-operation. In 4 h after operation, sacrifice the rats, anatomize and separate each part of the prostate, weigh wet weight, and compare swelling extents between the dose groups and the model group. Take the abdominal part of the prostate of rats, fix with 10% formalin, dehydrate routinely, imbedded with paraffin, slice up, conduct HE staining, and observe pathological changes under a microscope.

Observation criteria for pathology of prostatic tissue from rats of each group: \textcircled{1} Score of stroma swelling or blood vessel hyperemia: no stroma swelling or blood vessel hyperemia, 0; mild stroma swelling or blood vessel hyperemia, 1; moderate stroma swelling or blood vessel hyperemia, 2; severe stroma swelling or blood vessel hyperemia, 3; \textcircled{2} Score of infiltration of inflammatory cells: no stroma inflammatory cells, 0; small amount of stroma inflammatory cells, 1; moderate amount of stroma inflammatory cells, 2; high amount of stroma inflammatory cells, 3; stroma diffuse inflammatory cells, 4.

The experiment showed that, in 4 h after inflammation caused by carrageenan, severe swelling and obvious inflammation occurs in the prostate of rats. Through statistic analysis, the weight of the abdominal part of the prostate increased very significantly (p<0.001) in rats of the model group compared with those of the controlled group; however, the weights of the back or side leaves of the prostate are not influenced significantly (p>0.05). In the high-, moderate-, and low-dose groups, and the indomethacin group, compared with the model groups, EOB-f01 obviously inhibited the increase of weight of the abdominal part of the prostate (p<0.001, p<0.01, p<0.05, and p<0.01), appearing dose-dependent; however, it did not influence significantly the back and side parts of the prostate; see Table 4.

TABLE 4
Effects of EOB-f01 on Prostate Weight Increase Caused by Carrageenan (X ± SD)

| | Dose | n | Prostate weights (mg) | | |
|---|---|---|---|---|---|
| Groups | (mg/kg) | (Count) | Abdominal part | Side part | Back part |
| Blank-controlled group | — | 10 | 274.0 ± 67.2 | 66.4 ± 12.0 | 122.7 ± 27.8 |
| Model group | — | 10 | 466.5 ± 79.1^{###} | 73.3 ± 17.3 | 122.1 ± 23.3 |
| Indomethacin group | 12 | 10 | 358.1 ± 79.2** | 62.0 ± 12.3 | 128.6 ± 20.9 |
| High-dose group | 400 | 10 | 309.1 ± 87.2*** | 60.5 ± 21.2 | 116.0 ± 17.8 |
| Moderate-dose group | 200 | 10 | 326.9 ± 84.4** | 65.6 ± 13.3 | 126.4 ± 27.2 |
| Low-dose group | 100 | 10 | 375.6 ± 80.9* | 68.7 ± 14.4 | 122.9 ± 18.9 |

^{###}p < 0.001, compared with the blank-controlled group;
***p < 0.001,
**p < 0.01,
*p < 0.05, compared with the model group.

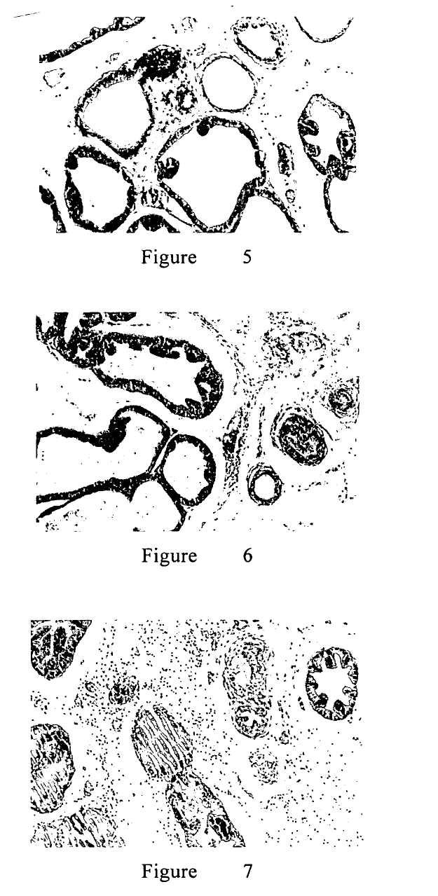

The histopathological study on the abdominal part of the prostate in rats showed: prostatic tissues of rats in the control group are normal, lobulated. And majority of the prostatic epithelium is columnar cells in single layer, having brush-like edge, and with papillary protuberances in part of the prostatic epithelium; light red secretions in part of the glands, small stroma between the glands, no inflammatory change and fibrous hyperplasia in stroma; there are similar structures in the abdominal part of the prostate in rats in the model group, all EOB-f01 groups and Indomethacin group: compared with that of rats in the controlled group, the prostatic epithelium slightly raised, multiple-layered; slight hyperplasia in glands, or papillary hyperplasia in part of the prostatic epithelium; dilation or swelling in blood vessels of stroma, accompanied with dispersive infiltration of high to low amount of neutrophils, no fibrous hyperplasia in stroma, showing as non-bacterial stroma inflammation. Inflammation was significantly decreased in rats in various EOB-f01 experimental groups compared with those in the controlled group. See Table 5 for score of histopathological observation data, and see attached FIGS. 4-7 for respective pathological slices.

TABLE 5
Histopathological Observation of EOB-f01 on Inflammation
of the abdominal Part of the Prostate in Rats Caused by
Carrageenan ( X̅ ± SD)

| | | | Degree of dilation | Degree of |
|---|---|---|---|---|
| | Dose | n | or swelling of | infiltration of |
| Groups | (mg/kg) | (Count) | stroma blood vessels | neutrophils |
| Blank-controlled | — | 10 | 0 | 0 |
| group | | | | |
| Model group | — | 10 | 2.6 ± 0.5^{###} | 2.6 ± 1.3^{###} |
| Indomethacin | 10 | 10 | 2.1 ± 2.0 | 2.2 ± 1.3 |
| group | | | | |
| High-dose group | 400 | 10 | 1.3 ± 1.3** | 1.4 ± 1.2* |
| Moderate-dose | 200 | 10 | 1.4 ± 0* | 1.4 ± 0.8* |
| group | | | | |
| Low-dose group | 100 | 10 | 1.4 ± 0* | 1.7 ± 1.2 |

^{###}p < 0.001, compared with the blank-controlled group;
*p < 0.05,
**p < 0.01, compared with the model groups.

3. Anti-Prostatic Hyperplasia

Randomly assign ICR mice, male, body weight 18-21g, into 7 groups, 10-11 mice in each group: (1) controlled group: water of equal volume, 0.8 mL/20 g, ig; (2) model group: 0.8 mL/20 g water of equal volume, ig; (3) estradiol group: estradiol 0.5 mg/kg, sc, once every 3 days, for 5 times; (4) high-dose group: EOB-f01 400 mg/kg ig; (5) moderate-dose group: EOB-f01 200 mg/kg, ig; (6) low-dose group: EOB-f01 100 mg/kg, ig. Once per day, for 14 days. Except the controlled group (1), various groups (2)-(6) with testosterone propionate 5 mg/mL per day, sc, for 14 d. On the fifteenth day, sacrifice the mice, anatomize them, take and weigh the abdominal, side, and back parts of the prostate, and evaluate anti-hyperplasia effects of EOB-f01.

The results showed that, there is significant increase of weights of the abdominal, side, and back parts of the prostate, and significant hyperplasia, in mice of the model group administered with testosterone propionate, compared with those in the controlled group. In mice in various experimental groups, compared with those in the model group, EOB-f01 significantly inhibited hyperplasia of the abdominal part of the prostate of mice in high- and moderate-dose groups (p<0.01 and p<0.05), but did not significantly influence the other parts of the prostate (p>0.05); however, estradiol significantly inhibited all parts of the prostate (p<0.001, and p<0.01); see Table 6.

TABLE 6
Effects of EOB-f01 on Prostatic Hyperplasia Caused by Testosterone
Propionate ( X̅ ± SD)

| | Dose | n | Prostate weights (mg) | | |
|---|---|---|---|---|---|
| Groups | (mg/kg) | (Count) | Abdominal part | Side part | Back part |
| Blank-controlled group | — | 10 | 7.6 ± 2.1 | 14.1 ± 5.2 | 5.8 ± 1.6 |
| Model group | 5 | 10 | 11.3 ± 2.8^{##} | 23.6 ± 6.2^{##} | 7.8 ± 1.8^{#} |
| Estradiol group | 5 + 0.5 | 10 | 7.0 ± 1.7*** | 15.1 ± 2.7*** | 5.1 ± 1.6** |
| High-dose group | 5 + 400 | 11 | 8.1 ± 2.1** | 22.7 ± 4.9 | 6.5 ± 1.5 |
| Moderate-dose group | 5 + 200 | 10 | 8.5 ± 1.9* | 23.9 ± 6.28 | 6.2 ± 2.1 |
| Low-dose group | 5 + 100 | 10 | 9.1 ± 1.5 | 22.4 ± 6.0 | 6.8 ± 2.0 |

^{##}p < 0.01,
^{#}p < 0.05, compared with the blank-controlled group;
***p < 0.001,
**p < 0.01,
*p < 0.05, compared with the model group.

Prostatic hyperplasia is a common disease in senile males; epidemiological studies have demonstrated that, prostatic hyperplasia occurs only in senile males with normal testis function, and its pathogenesis is not clearly known so far. Most researchers consider that, disorders in equilibrium of sex hormone or relevant hormone secretions are the main basis for its occurrence. The presence of androgen is the basis for development of the prostate, and the primary cause for its hyperplasia; and estradiol may increase proliferation of prostatic matrix in low dose, but decrease proliferation of it in high dose. EOB-f01 significantly inhibits growth of the abdominal part of the prostate of young mice in high or moderate doses, but does not influence growth of seminal vesicle, elevator ani muscle, and testis, suggesting that EOB-f01 may have direct effects on the prostate. Meanwhile, EOB-f01 inhibits hyperplasia of the abdominal part of the prostate of mice, caused by testosterone propionate, suggesting that EOB-f01 inhibits prostatic hyperplasia caused by sex hormones.

4. Effects on Growth of the Prostate and the Accessorial Sex Organs of Young Mice

Assign ICR young male mice, body weight 10-12 g, into groups according to their body weight, 10-11 mice in each group: (1) controlled group: water of equal volume, 0.8 mL/20 g, ig; (2) estradiol group: estradiol 0.5 mg/kg, sc, once every 3 days, for 5 times; (3) high-dose group: EOB-f01 400 mg/kg, ig; (4) moderate-dose group: EOB-f01 200 mg/kg, ig; (5) low-dose group: EOB-f01 100 mg/kg, ig. Once per day, for 14 days. On the fifteenth day, sacrifice the mice, anatomize and weigh the abdominal, side, and back parts of the prostate, and seminal vesicle, testis, and elevator ani muscle. The results showed that, EOB-f01 significantly inhibits growth of the abdominal part of the prostate of young mice in high- and moderate-dose groups (p<0.05), but did not significantly influence that of the side and back parts of the prostate (p>0.05); however, estradiol significantly inhibits growth of all parts of the prostate of young mice; see Table 7.

TABLE 7
Effects of EOB-f01 on Growth of the Prostate of Young Mice ( X̅ ± SD)

| | Dose | N | Prostate weights (mg) | | |
|---|---|---|---|---|---|
| Groups | (mg/kg) | (Count) | Abdominal part | Side part | Back part |
| Blank-controlled group | | 11 | 6.7 ± 1.1 | 9.6 ± 3.3 | 4.2 ± 1.5 |
| Estradiol group | 0.5 | 10 | 1.7 ± 0.5*** | 1.5 ± 1.3*** | 1.1 ± 0.9*** |
| High-dose group | 400 | 10 | 5.0 ± 1.7* | 8.1 ± 4.4 | 3.2 ± 1.1 |
| Moderate-dose group | 200 | 11 | 5.2 ± 1.5* | 10.0 ± 3.8 | 3.8 ± 1.5 |
| Low-dose group | 100 | 10 | 5.9 ± 1.6 | 8.9 ± 3.3 | 4.0 ± 1.5 |

***p < 0.001,
*p < 0.05, compared with the blank-controlled group.

EOB-f01 significantly decrease weight of seminal vesicle of young mice in high-dose group, but did not significantly influence weights of seminal vesicle, testis, and elevator ani muscle of young mice in the other groups; however, estradiol significantly inhibits growth of seminal vesicle, testis, and elevator ani muscle of young mice (p<0.01); see Table 8.

TABLE 8
Effects of EOB-f01 on Growth of Seminal Vesicle, Testis, and Elevator Ani
Muscle ( X̅ ± SD)

| | Dose | | Testis | Seminal vesicle | Elevator ani |
|---|---|---|---|---|---|
| Groups | (mg/kg) | n (Count) | (mg) | (mg) | muscle (mg) |
| Blank-controlled group | | 11 | 151.3 ± 28.2 | 34.1 ± 11.3 | 42.5 ± 10.6 |
| Estradiol group | 0.5 | 10 | 58.0 ± 12.8*** | 7.3 ± 2.5*** | 14.1 ± 3.3*** |
| High-dose group | 400 | 10 | 140.0 ± 30.9 | 20.5 ± 8.8** | 35.6 ± 8.3 |
| Moderate-dose group | 200 | 11 | 158.9 ± 14.1 | 33.5 ± 12.0 | 47.1 ± 12.8 |
| Low-dose group | 100 | 10 | 141.4 ± 14.8 | 33.9 ± 10.8 | 43.4 ± 11.5 |

***p < 0.001,
*p < 0.01, compared with the blank-controlled group.

5. Anti-Platelet Aggregation

In 2001, the applicant again entrusted Shenyang Pharmaceutical University to conduct experiment of EOB-f01 on anti-platelet aggregation. In Tables 9 and 10, it is obvious that EOB-f01 has the effect of anti-platelet aggregation in rabbits in vivo and in vitro, significantly compared with those of the negative group; in in-vivo experiment on anti-platelet aggregation, the effects of anti-platelet aggregation is stronger in moderate- and high-dose groups than in the compound salvia tablets group.

TABLE 9
Effects of EOB-f01 on in vitro Platelet Aggregation in Rabbits
( X̅ ± SD, n = 8)

| | | Count | | Rate of |
|---|---|---|---|---|
| | Doses | of | Rate of platelet | inhibition |
| Groups | (mg/kg) | animals | aggregation (%) | (%) |
| Controlled group | — | 8 | 36.36 ± 6.65 | — |
| High-dose group | 10 | 8 | 10.97 ± 4.70** | 69.82 |
| Moderate-dose group | 5 | 8 | 17.13 ± 3.25* | 52.88 |
| Low-dose group | 2.5 | 8 | 21.91 ± 2.05* | 39.74 |

**p < 0.01,
*p < 0.05, compared with the controlled group.

TABLE 10
Effects of EOB-f01 on in-vivo Platelet Aggregation in
Rabbits ( X̅ ± SD, n = 8)

| | | Count | | |
|---|---|---|---|---|
| | Doses | of | Rate of platelet | Rate of |
| Groups | (g/kg) | animals | aggregation (%) | inhibition (%) |
| Controlled group | — | 8 | 51.17 ± 4.96 | — |
| High-dose group | 0.4 | 8 | 27.27 ± 4.85*** | 46.57 |
| Moderate-dose | 0.2 | 8 | 31.66 ± 2.64*** | 38.13 |
| group | | | | |
| Low-dose group | 0.1 | 8 | 38.63 ± 2.65** | 24.51 |
| Compound salvia | 0.1 | 8 | 35.67 ± 6.67** | 30.30 |
| tablets group | | | | |

***P < 0.001,
**P < 0.01,
*P < 0.05, compared with the controlled group.

6. Promotion of Immunity

Samples of Lophatherum Total Flavones: product EOB-f03, content of standard total flavones glycoside ≧10%, experimental value 13.6%, light brown powder, obtained from Hangzhou Lifu Bio-science and Technology Co., Ltd., of Zhejiang University. Male mice of Kunming strain, body weight 18-22 g, obtained from the Experiment Center of Nanjing Railway Hospital, were assigned randomly into the negative control group, and low-, moderate-, and high-dose group, corresponding to 5, 10, and 30 times the recommend dose of human body, respectively. The experiment was carried out according to the specified method for immune adjustment in the Evaluation Procedures and Inspection Methods for Function of Health Food, issued by the Supervising Department of the Ministry of Public Health.

6.1 Effects on Production of Serum Hemolysin Antibody of Mice

The content of serum hemolysin antibody of mice was expressed as HC₅₀ of samples; the experiment showed (Table 11) that, there is significant difference between the moderate-dose and controlled groups (p<0.05), and extremely significant difference between the high-dose and controlled groups (p<0.01), suggesting that EOB-f03 may promote production of antibody in mice, and enhance immunity of body fluid.

TABLE 11
Effects of EOB-f03 on Serum Hemolysin of Mice ( X̅ ± SD)

| Groups | Doses (mg/kg) | n (Count) | HC₅₀ |
|---|---|---|---|
| Controlled group | — | 10 | 132.06 ± 24.77 |
| High-dose group | 900 | 10 | 155.21 ± 3.79** |
| Moderate-dose | 300 | 10 | 152.04 ± 4.51* |
| group | | | |
| Low-dose group | 150 | 10 | 150.42 ± 15.02 |

*P < 0.05,
**P < 0.01, compared with the controlled group.

6.2 Effects on the Expurgation Speed of Carbon Particles in Mice

TABLE 12
Effects of EOB-f03 on Expurgation Speed of Carbon Particles
in Mice ( X̅ ± SD)

| Groups | Doses (mg/kg) | n (Count) | Phagocytosis index (a) |
|---|---|---|---|
| Controlled group | — | 10 | 2.33 ± 0.96 |
| High-dose group | 900 | 10 | 3.71 ± 1.14** |
| Moderate-dose | 300 | 10 | 3.2 ± 0.69* |
| group | | | |
| Low-dose group | 150 | 10 | 2.77 ± 1.12 |

*P < 0.05,
**P < 0.01, compared with the controlled group.

Conduct the one-factor variance analysis for phagocytosis index of each group in Table 12, F=3.491, p<0.01; there are significant differences between means of phagocytosis indexes of different dose groups; the results of comparison between any two groups showed that, there are significant differences between the groups of more than 300 mg/kg EOB-f03 and the controlled group. It suggested that, EOB-f03 may dramatically increase phagocytosis of macrophages in mice.

6.3 Effects on Delayed Allergy of Mice Induced by DNFB

TABLE 13
Effects of EOB-f03 on Delayed Allergy (DTH) in Mice ( X̅ ± SD)

| | Doses | n | Weight difference of earlaps |
|---|---|---|---|
| Groups | (mg/kg) | (Count) | (mg) |
| Controlled group | — | 10 | 7.10 ± 2.69 |
| High-dose group | 900 | 10 | 9.30 ± 1.70* |
| Moderate-dose group | 300 | 10 | 9.20 ± 1.32* |
| Low-dose group | 150 | 10 | 7.20 ± 2.49 |

*P < 0.05, compared with the controlled group.

Conduct the one-factor variance analysis for weights of left and right earlaps, F=3.004, p<0.05, and there are significant differences between weights of different dose groups; Compare the weight difference of left and right earlaps in different dose groups, the weight differences of the high- and moderate-dose groups are significant. It suggested that EOB-f03 may promote cellular immunity of mice.

7. Anti-Tumor Effects

Since 2002, based on EOB-f01, the inventor utilized High-Speed Countercurrent Chromatography (HSCCC), and obtained two C-glycoside flavonoid mixtures with more than 95% of the content of orientin and homoorientin, and performed the screening for anti-tumor effects in vitro; it showed that the two mixtures may inhibit to different extents the growth of the cancer cells of DU145 (human prostate cancer cellular strain), P₃₈₈ (mouse leukemia), A₅₄₉ (human adenocarcinoma of lung), A₃₇₅ (human melanoma), L₉₂₉ (mouse lung epithelium carcinoma), Hela (human cervical cancer), and THP-1 (human macrophage tissue tumor), with inhibition rates appearing time- and dose-dependent.

8. Clinical Observation

During consumption of Zhukangning capsules (Health Food Healthcare [1999] No. 0564; approved healthcare functions: adjustment of blood lipid, improvement of immunity; with core of EOB-f03, 250 mg/capsule, and content of total flavones glycosides 10%), unexpected effects were found in many consumers stubbornly with prostatic hyperplasia and prostatitis. Examples are as follows:

Gan xx, male, 73 years old, lived in Zhejiang Ningbo, a retired cadre, with a history of family hypertension, and with higher level of blood lipid. For more than 20 years, he had taken chronically antihypertensive drugs including nifedipine tablets and compound captopril tablets, as well as Fufang Jiangya tablets, Fufang Luobuma tablets, and Kaibotong, etc., and hypolipidemics including Yuyou Jiangzhi pills, Xiezhiqing, and Duoxikang, etc., assisted with triprolidine hydrochloride, gingko biloba tablets, and Tianbaoning, etc. But it still did not achieve satisfactory effects. His blood pressure may reach as high as 220/120 mmHg, and 180/90 mmHg in general, and both his blood triglycerides and Cholesterol exceed the normal range. Since Jul. 7, 1998, he insisted on taking nifedipine, and adding Zhukangning capsules instead of Yuyou Jiangzhi pills and gindgo biloba tablets, 3 capsules, twice per day, for 2 months. After one week of withdrawal, he was rechecked on September 14, blood pressure 170/85 mmHg, and various indexes of blood lipids recovering to normal (TG decreased from 1.83 to 0.90 mmol/L, TC decreased from 5.90 to 4.82 mmol/L, apolipoprotein A increased from 1.26 to 1.67 g/L, and apolipoprotein B decreased from 1.14 to 1.00 g/L). After taking Zhukangning capsules for more than one month, he accidentally found he needed get up just once for urine while 3-4 times before, suggesting significant effects.

Hou x, male, 74 years old, lived in Hunan Changsha, a retired cardre. He suffered a heavy cold in 1986, with body discomfort for more than one month, and then found abnormal urination; confirmed as suffered from prostatitis through inspection in hospital, with main symptoms as follows: at the time of the first urination in the morning, with very strong cauterant feeling in urethra; incomplete in routine urination, urine flow discontinuous like line, and pain at urination. He had sought doctors for more than 10 years, purchased drugs through mail order, and spent much money without any effects; his conditions are gradually exacerbated with intolerable pain. Since June 1999, he began to take Zhukangning capsules, insisted for approximately half a year, and found the above symptoms relieved significantly, with life quality increased greatly.

### CONCLUSION

As the pathogenesis of prostatic diseases is not clearly known, we conducted the above-mentioned experiments according to its pathological features and possible pathogenesis, and then we elucidated the potential effects of EOB-f in prevention and treatment of prostatitis, prostatic hyperplasia, and prostate cancer, suggesting its potential of development as a second-line therapy for prostatis diseases or as a supplement of health food.

Lophatherum Total Flavones (EOB-f), indicated by this invention, are flavone preparations of different purity, obtained from leaves of graminae, bambusoideae, or phylostachy sieb. Et zucc, with its manufacturing process involved in the two patents of invention (patent numbers are ZL 98 1 04564.2 ZL 98 1 04563.4, respectively). It shall be indicated that, Lophatherum Total Flavones, indicated in those patents, are either products obtained through the manufacturing process of the above-mentioned patents, or based on those patents, lophatherum flavone products obtained through techniques including absorption-desorption, column chromatography, membrane separation, recrystallization, and chromatographic separation, etc., or through the combination thereof.

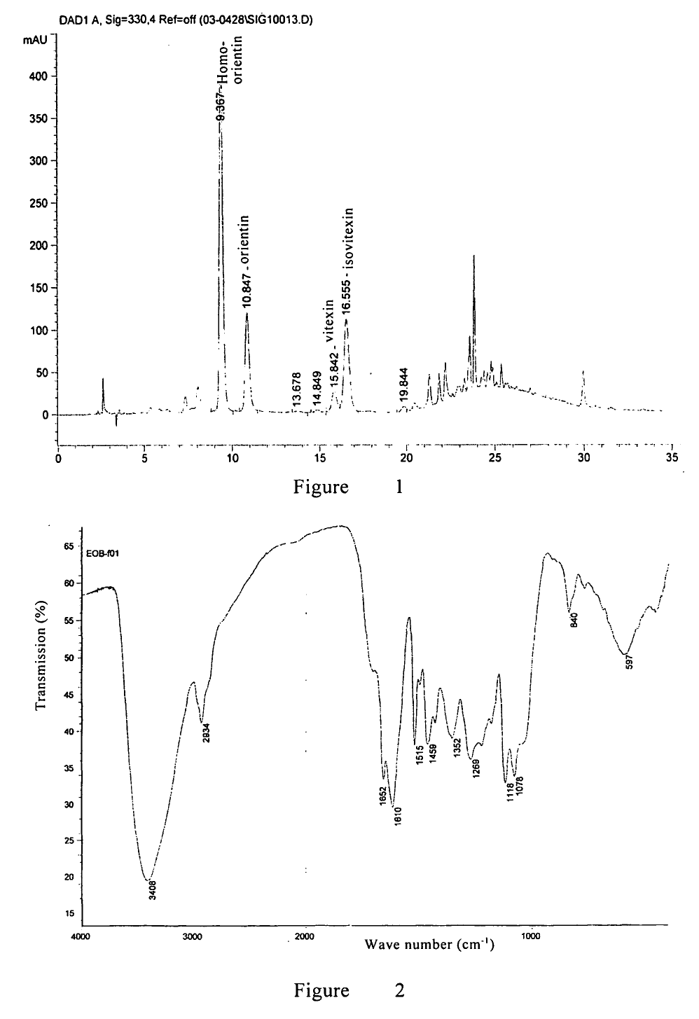

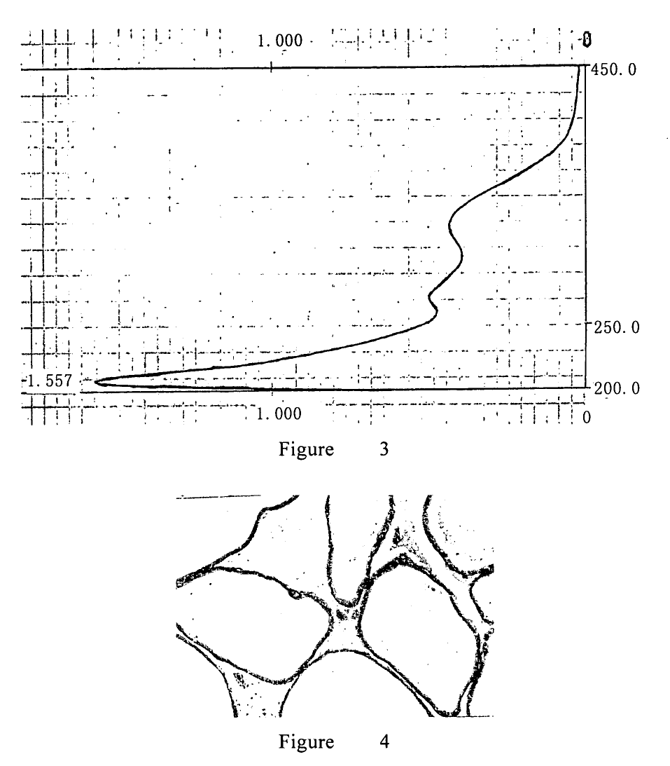

The appearance of Lophatherum Total Flavones are yellow or light brown powder (or extract); content of total flavones glycoside, 10-90% (aluminum nitrate-sodium nitrite, rutin as the standard, calculated in dry basis); see FIG. 1 for the four main C-glycosides, i.e. orientin, homoirientin, vitexin, and isovitexin. Infra-red chromatography of it, tabletted by potassium bromide, showed that, Lophatherum Total Flavones have characteristic absorptions at 3408, 2934, 1652, 1610, 1118, and 1078 cm⁻¹ (FIG. 2); when it dissolved in spectroscopically pure methanol, the solution was scanned at 200-600 nm, and the chromatogram showed there is a strong absorption peak at 240-280 nm, and another strong absorption peak at 300-350 nm, complying with typical features of Lophatherum Total Flavones (FIG. 3).

Identification of Lophatherum Total Flavone (EOB-f) by chemical reagents: Dissolve 0.5 g sample in 95% ethanol 100 mL to produce a solution; \textcircled{1} add 1% FeCl₃ 2-3 drops to the above solution 1 mL and it shall appear deep blue or blue violet. \textcircled{2} Add 1% AlCl₃-ethanol solution 2-3 drops to the above solution 1 mL, and it shall appear bright yellow. \textcircled{3} Add ether 10 mL to the sample 0.5 g, extract for 30 s assisted with ultrasonic wave, and then filter. Place the filtrate 1 mL on a water bath at 70-90° C., and evaporate ether to dry, and add successively 2% m-dinitrobenzene (in 95% ethanol) and 2.5 mol/L KOH, 1 ml each, respectively; it shall appear slight red, and immediately become deep violet-red after placed on the above water bath.

The latest studies, involved in this invention, showed that, Lophatherum Total Flavones have: certain inhibition against 7 common pathogenic bacteria to urinary system; obvious inhibition for ear-inflammation models of mice, caused by croton oil and xylene; obvious inhibition for prostatitis model of rats, caused by carrageenan; certain inhibition against growth of the prostate of young mice, but no obvious influence against growth of testis, seminal vesicle, and elevator ani muscle; obvious inhibition against prostatic hyperplasia of mice, caused by testosterone propionate; obvious anti-platelet aggregation in in-vitro and in vivo experiments of rabbits; obvious promotion for humoral and cellular immunity of mice, and for phagocytosis of macrophages; and inhibition against hyperplasia of a variety of tumor cellular strains; Lophatherum Total Flavones have potential of development as a natural drug for prevention and treatment of prostatic diseases, and as a supplement of health foods.

Health foods or natural drugs for prevention and treatment of prostatic diseases, indicated in this invention, may use Lophatherum Total Flavones alone as active ingredient or functional factor, or in combination with other Chinese or Western bulk drugs or vegetable extracts, generally used as drugs for prostatic diseases; those other ingredients may includes: various antibiotics, α-receptor blockers, anti-inflammatory drugs, or extracts from other species plants (such as vegetative polysaccharides, vegetative flavones, vegetative sterols, pollen extracts, or lycopene, etc.).

As an active ingredient, the dose (calculated on content of total flavones glycoside) of Lophatherum Total Flavones (EOB-f) is: for adults, 10-1000 mg per day, preferred 50-600 mg, once or twice per day.

The product of this invention may be manufactured in many forms including tablets, capsules, granules, pills, droplets, pre-emulsion, micro-emulsion, suspension, syrup, various enteric-coated forms, injections, sprays, or ointments, or preparation for intrarectal administration such as suppositories, etc.

Drugs and/or health products of this invention have effects on prevention and treatment of disorders of urinary system, such as prostatitis (bacterial or non-bacterial), prostatic hyperplasia, as well as prostate cancer.

The advantages of this invention: provide a vegetative extract with extensive sources, safety and effectiveness, and economy and applicability—Lophatherum Total Flavones (EOB-f), and conduct systemic investigation on its features related with prostate pathology, suggesting that it has significant effects of anti-bacteria, anti-inflammation, anti-prostatic hyperplasia, anti-platelet aggregation, anti-tumor, and immunity promotion; lots of previous studies have demonstrated that, it has excellent physiological and pharmacological effects of anti-free radicals, anti-oxidation, anti-radiation, and cadio- or cerebro-vascular protection, etc., with safety and without toxicity, and without any side effects in chronic administration; moreover, it has stable property and fragrant taste, and may be manufactured in various forms, or in combination with many drugs and foods, ready for use, especially for prevention and treatment of prostatic diseases, a type of senile chronic degenerative diseases with multiple targets.

This invention is described through the following non-restrictive examples. Unless indicated, percentage is weight percentage.

### Example 1

Film-coated tablets, 75 mg/tablet (calculated on total Flavones glycoside)

Active ingredients: Lophatherum Total Flavones (EOB-f01 power, content of total flavones glycoside ≧50%)

Excipients: internally filled auxiliary materials, including micro-crystalline cellulose, L-hydroxypropylcellulose (L-HPC), sodium dodecylsulfate, and magnesium stearate. Compositions of film coat: corn protein, ethylcellulose, plasticizers, pharmaceutical pigments, and titanium dioxide, etc.

Manufactured in common or heterotypic tablets, according to requirements. With regard to symptoms, use it as prescribed or according to instruction, 1-4 tablets each, once or twice per day.

### Example 2

Enteric-coated tablets, 50 mg/tablet (calculated on total flavones glycoside)

Active ingredients: Lophatherum Total Flavones (EOB-f01 power, content of total flavones glycoside ≧50%)

Excipients: internally filled auxiliary materials, including micro-crystalline cellulose, polyethylene, Polyvinylpyrolidone, sucrose sulfate, and magnesium stearate. Composition of enteric coat: poly(acrylic acid) II and/or III resin, Tween 80, diethyl dibenzoate, diethyl phthalate, caster oil, and pharmaceutical pigments, etc.

Manufactured in common or heterotypic tablets, according to requirements. With regard to symptoms, use it as prescribed or according to instruction, 1-4 tablets each, once or twice per day.

### Example 3

Capsules 100 mg (calculated on total flavones glycoside)

Active ingredients: Lophatherum Total Flavones (EOB-f01 power, content of total flavones glycoside ≧50%)

Excipients: internally filled auxiliary materials, including lactose, carboxymethylcellulose (CMC), sodium dodecylsulfate, and talc powder. Composition of outer shell of capsule: gelatin, pharmaceutical pigments, and titanium dioxide, etc.

Manufactured in common or heterotypic tablets, according to requirements. With regard to symptoms, use it as prescribed or according to instruction, 1-3 capsules each, once or twice per day.

### Example 4

Pre-microemulsion, 50 mg/mL (calculated on total flavones glycoside)

Active ingredients: Lophatherum Total Flavones (EOB-f01 power, content of total flavones glycoside ≧50%)

Excipients: internally filled auxiliary materials, including non-ionic surface active-agents, auxiliary solvent, liposoluble emulsion, plant oils, and anti-oxidants.

Manufactured in oral solution or soft capsules, according to requirements. With regard to symptoms, use it as prescribed or according to instruction, 1-4 mL (or capsules), once or twice per day.

### Example 5

Injection, 2 mg/mL (calculated on total flavones glycoside)

Active ingredients: Lophatherum Total Flavones (EOB-f01 power, content of total flavones glycoside ≧50%)

Excipients: Tween-80, sodium chloride, and water for injection.

Manufactured in 2 mL/vial, 5 mL/vial, or 10 mL/vial, according to requirements. With regard to symptoms, use it as prescribed or according to instruction.

### Example 6

Suppositories, 20 mg/g (calculated on total flavones glycoside)

Active ingredients: Lophatherum Total Flavones (EOB-f01 power, content of total flavones glycoside ≧50%)

Excipients: internally filled auxiliary materials, including Witepsol H15, Witepsol W45, and sodium dodecylsulfate, etc.

Manufactured in 2 g/suppository, according to requirements. With regard to symptoms, use it as prescribed or according to instruction, 1-4 times per day.

### Example 7

Compound film-coated tablets, 50 mg (calculated on total flavones glycoside) plus Finasteride 2 mg

Active ingredients: Lophatherum Total Flavones (EOB-f01 power, content of total flavones glycoside ˜50%) and finasteride (a chemical synthetic drug)

Excipients: internally filled auxiliary materials, including microcrystalline cellulose, L-hydroxypropylcellulose (L-HPC), sodium dodecylsulfate, and magnesium stearate. Compositions of film coat: corn protein, ethylcellulose, plasticizers, pharmaceutical pigments, and titanium dioxide, etc.

Manufactured in common or heterotypic tablets, according to requirements. With regard to symptoms, use it as prescribed or according to instruction, 1 tablet each, once or twice per day.

### Example 8

Compound capsules, 40 mg (calculated on total flavones glycoside) plus lycopene oil resin 8 mg

Active ingredients: Lophatherum Total Flavones (EOB-f01 power, content of total flavones glycoside ≧50%) and lycopene oil resin (containing lycopene 8%)

Excipients: internally filled auxiliary materials, including lactose, carboxymethylcellulose (CMC), sodium dodecylsulfate, and talc powder. Composition of outer shell of capsule: gelatin, pharmaceutical pigments, and titanium dioxide, etc. Manufactured in common or heterotypic tablets, according to requirements. With regard to symptoms, use it as prescribed or according to instruction, 1-3 capsules each, once or twice per day.

All literatures mentioned in this invention, are incorporated herein by reference, as each literature is incorporated separately by reference. In addition, it shall be understood that, after reading the above teachings of this invention, a skilled in the art will be able to made various changes or modifications to this invention and these equivalents shall fall into the ranges limited by the attached claim of this application.

1. A use of Lophatherum Total Flavones in medicine and health food as a factor for prevention and treatment of prostatic diseases, wherein the use is in the manufacture of medicine and health food for prevention and treatment of prostatic diseases.

2. The use according to claim 1, wherein the prostatic diseases comprises prostatitis, prostatic hyperplasia, and prostate cancer.

3. The use according to claim 1, wherein the medicine and health food comprise Lophatherum Total Flavones and a substance for prevention and treatment of prostatic diseases which is selected from the group consisting of: antibiotics, α-receptor blockers, anti-inflammatory drugs, and plant extracts.

4. The use according to claim 3, wherein the forms of medicine and health food product are tablets, capsules, granules, pills, droplets, pre-emulsion, micro-emulsion, suspension, syrup, various enteric-coated forms, injections, sprays, ointments, or preparations for intrarectal administration such as suppositories.

5. The use according to claim 4, wherein the dose of Lophatherum Total Flavones (content of total flavones glycoside, calculated on rutin) is 10-1000 mg/day for adults, once or twice per day.

6. The use according to claim 4, wherein the preferred dose of Lophatherum Total Flavones (content of total flavones glycoside, calculated on rutin) is 50-600 mg/day for adults, once or twice per day.

7. The use according to claim 3, wherein the plant extracts are selected from the group consisting of vegetable polysaccharides, vegetable flavones, vegetable sterols, pollen extracts, and lycopene.

8. The use according to claim 1, wherein the medicine and health food comprise 0.01-99 wt % of Lophatherum Total Flavones.

9. The use according to claim 1, wherein the medicine and health food comprise 0.1-90 wt % of Lophatherum Total Flavones.
